(19) United States
(12) Patent Application Publication
(10) Pub. No.: US 2026/0128167 A1
(43) Pub. Date: May 7, 2026

(54) MEDICAL DEVICE APPLICATION MANAGEMENT

(71) Applicant: Welch Allyn, Inc., Skaneateles Falls, NY (US)

(72) Inventors: Matthew J. Winter, Syracuse, NY (US); Nicholas J. Fabbricante, New Gloucester, ME (US); Christopher M. Haigney, Syracuse, NY (US); Christopher M. Keegan, Marcellus, NY (US); Michael E. Rice, Liverpool, NY (US)

(21) Appl. No.: 18/935,155

(22) Filed: Nov. 1, 2024

(51) Int. Cl. G16H 40/63; G06F 3/0482; G06F 9/451

(52) U.S. Cl. CPC G16H 40/63; G06F 9/451; G06F 3/0482

(57) ABSTRACT

A system for managing medical device applications. The system provides a user interface engine and receives a custom resource definition of the user interface engine for an application configured for execution on a medical device. The system processes the custom resource definition, launches a configuration portal, and dynamically renders a user interface in the configuration portal. The user interface is rendered based on the custom resource definition. The system receives configuration inputs on the user interface, and persist the configuration inputs into a catalogue database that causes the application to be executed on the medical device based on the configuration inputs.

### BACKGROUND

The management of software applications for medical devices is a critical component of modern healthcare delivery, encompassing the development, deployment, and maintenance of software that is either embedded in or intended to be used with medical devices. This management ensures that software applications meet regulatory requirements, function as intended, and provide secure and effective operation throughout their lifecycle.

Software applications for medical devices are often updated to work seamlessly with other systems and devices within a healthcare environment. Interoperability ensures that data can be shared and utilized effectively, improving patient care and clinical decision-making. Also, software applications for medical devices often require regular updates to correct any defects, improve functionality, and address security vulnerabilities. Maintenance also involves ensuring compatibility with updated hardware or other software systems.

The management of software applications for medical devices is a comprehensive and dynamic process that is driven by the need to ensure interoperability, patient safety, data security, and regulatory compliance, while also fostering innovation and the efficient use of technology in healthcare. As medical devices become more software-driven and interconnected, the importance of robust software management practices will continue to escalate.

### SUMMARY

In general terms, the present disclosure relates to managing medical device applications. In one possible configuration, an application configuration registration custom resource is defined for dynamically rendering a user interface in a configuration portal that can be used to configure an application for use on one or more medical devices. Various aspects are described in this disclosure, which include, but are not limited to, the following aspects.

One aspect relates to a system for managing medical device applications, the system comprising: at least one processing device; and a memory device storing instructions which, when executed by the at least one processing device, cause the at least one processing device to: provide a user interface engine; receive a custom resource definition of the user interface engine for an application configured for execution on a medical device; process the custom resource definition; launch a configuration portal; dynamically render a user interface in the configuration portal, the user interface being rendered based on the custom resource definition; receive configuration inputs on the user interface; and persist the configuration inputs into a catalogue database that causes the application to be executed on the medical device based on the configuration inputs.

Another aspect relates to a method of managing medical device applications, the method comprising: providing a user interface engine; receiving a custom resource definition of the user interface engine for an application configured for execution on a medical device; processing the custom resource definition; launching a configuration portal; dynamically rendering a user interface in the configuration portal, the user interface being rendered based on the custom resource definition; receiving configuration inputs on the user interface; and persisting the configuration inputs into a catalogue database that causes the application to be executed on the medical device based on the configuration inputs.

Another aspect relates to a computer-readable non-transitory storage media embodying software that is operable when executed to: provide a user interface engine; receive a custom resource definition of the user interface engine for an application configured for execution on a medical device; process the custom resource definition; launch a configuration portal; dynamically render a user interface in the configuration portal, the user interface being rendered based on the custom resource definition; receive configuration inputs on the user interface; and persist the configuration inputs into a catalogue database that causes the application to be executed on the medical device based on the configuration inputs.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for managing medical device applications that includes a connectivity platform communicatively coupled to a plurality of medical devices and a workstation computer.

FIG. 2 illustrates an example of a patient monitoring device that can be included in a fleet of medical devices in the system of FIG. 1.

FIG. 3 illustrates an example of a communications device that can be included in another fleet of medical devices in the system of FIG. 1.

FIG. 4 schematically illustrates an example of the connectivity platform of FIG. 1 communicatively coupled over a network to the patient monitoring device of FIG. 2.

FIG. 5 schematically illustrates aspects of the connectivity platform of FIG. 1.

FIG. 6 schematically illustrates an example of a method of managing medical device applications that can be performed by the connectivity platform of FIG. 1.

FIGS. 7-37 provide examples of a user interface language specification that can be used to construct custom resource definitions of an application configuration registration (ACR) by the connectivity platform of FIG. 1.

FIG. 38 illustrates an example of the user interface that can be dynamically rendered in a configuration portal during an operation of the method of FIG. 6 by using the examples of the user interface language specification shown in FIGS. 7-37.

FIG. 39 illustrates another example of a user interface that can be dynamically rendered in the configuration portal during the operation of the method of FIG. 6.

FIG. 40 illustrates an example of a drop down menu for defining an outbound connection type in a data field of the user interface shown in FIG. 39.

### DETAILED DESCRIPTION

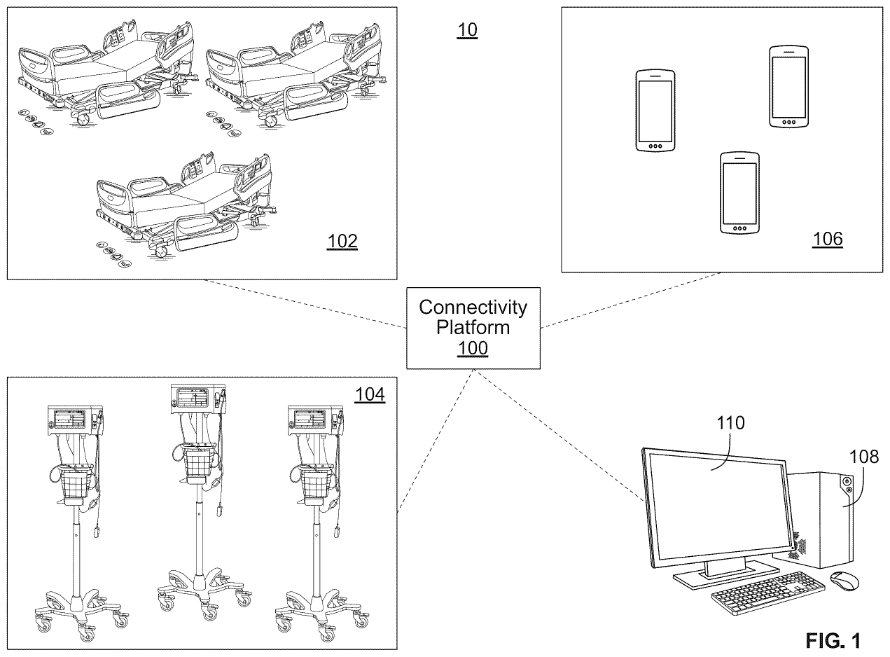

FIG. 1 schematically illustrates an example of a system 10 for managing medical device applications that includes a connectivity platform 100 that is communicatively coupled to a plurality of medical devices and a workstation computer 108. The connectivity platform 100 provides infrastructure that can be used to build applications for execution on one or more of the plurality of medical devices. For example, the connectivity platform 100 enables the applications installed on the medical devices to utilize one or more databases, single sign-on (SSO) authentication scheme, communications with electronic medical records (EMRs), and other types of infrastructure and services made available by the connectivity platform 100.

Additionally, the connectivity platform 100 provides a configuration portal that can be used to define and/or adjust one or more configurable settings of the applications for execution on one or more of the medical devices such as during a customer integration. As will be described in more detail, the connectivity platform 100 allows multiple applications to be configured in a single configuration portal. Further, the connectivity platform 100 does not need to be updated to accommodate changes to the configuration data of an existing application.

The connectivity platform 100 defines a user interface language for building the applications. The connectivity platform 100 further includes an engine that processes the user interface language to dynamically render a user interface in the configuration portal that can be displayed on a display monitor 110 of the workstation computer 108. The user interface displayed in the configuration portal by the connectivity platform 100 can be dynamically updated to display different configuration options for the applications installed on the medical devices within the system 10 without having to update or modify the connectivity platform 100. Advantageously, the applications and the connectivity platform 100 operate asynchronously.

In the example illustrated in FIG. 1, the system 10 includes different fleets of medical devices. As used herein, a fleet of medical devices means a grouping or a plurality of a certain type of medical device. As shown in FIG. 1, the system 10 includes a first fleet of medical devices 102 having one or more hospital beds, a second fleet of medical devices 104 having one or more patient monitoring devices, and a third fleet of medical devices 106 having one or more communications devices for providing communications between caregivers.

The hospital beds in the first fleet of medical devices 102 can each include one or more sensors such as load cells that detect the weight distribution and movements of a patient while resting on the hospital bed. The load cells can include one or more piezoelectric pressure sensors, strain gauges, and other types of sensors that can be positioned under a mattress where the patient rests to measure patient weight distribution and motion. The one or more sensors on the hospital beds can further include sensors for monitoring one or more physiological variables such as heart rate and respiration rate. The hospital beds can share aspects of the patient support apparatuses described in U.S. patent application Ser. No. 18/438,969, filed Feb. 12, 2024, entitled PATIENT SUPPORT APPARATUS HAVING VITAL SIGNS MONTORING AND ALERTING, the disclosure of which is herein incorporated by reference in its entirety.

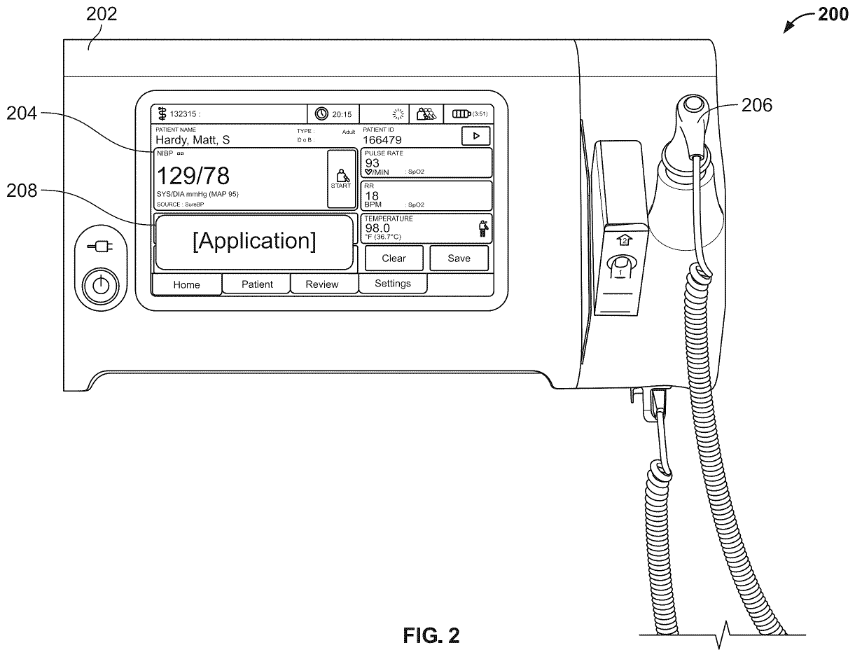

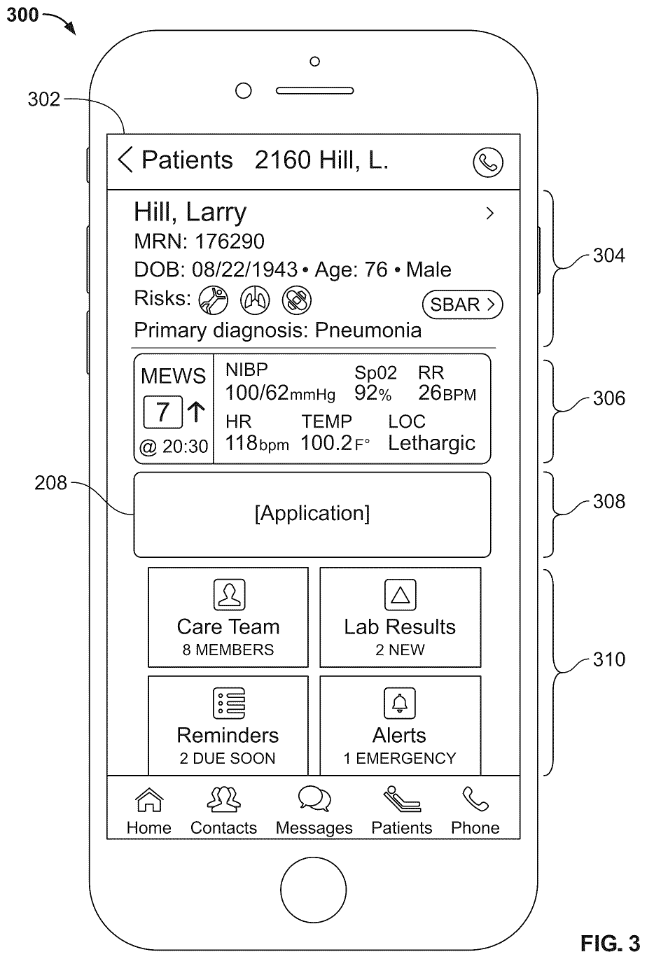

The patient monitoring devices in the second fleet of medical devices 104 include spot monitors that include one or more sensors for capturing physiological variables. An example of a patient monitoring device 200 in the second fleet of medical devices 104 of the system 10 is described in more detail further below with reference to FIG. 2. An example of a communications device 300 in the third fleet of medical devices 106 of the system 10 is described in more detail further below with reference to FIG. 3.

FIG. 1 is provided by way of illustrative example such that the system 10 can include additional types of medical devices or fewer types of medical devices based on the needs of a healthcare facility where the system 10 is implemented. Further, each of the fleets of medical devices can include one or more models or versions of a particular type of medical device. For example, the first fleet of medical devices 102 can include one or more models or versions of the hospital beds. Similarly, the second fleet of medical devices 104 can include one or more models or versions of the patient monitoring devices and the third fleet of medical devices 106 can include one or more models or versions of the communications devices.

The workstation computer 108 is operated by a user who is trained to maintain the medical devices in the system 10 such as a biomedical equipment technician (“biomed”) or a service technician. The biomed or service technician can use the workstation computer 108 to perform tasks such as maintenance and repairs of the plurality of medical devices, and/or procurement of new medical devices, and/or training of healthcare professionals.

FIG. 2 illustrates an example of a patient monitoring device 200 that can be included in the second fleet of medical devices 104 in the system 10. The patient monitoring device 200 includes features such as quick and secure access with single sign-on; accurate and quick blood pressure measurement; accurate hypertension detection with blood pressure averaging; body temperature measurement with a configurable thermometry module; blood oxygen saturation (SpO2) measurement with a configurable pulse oximetry module; ability to spot-check respiration rate; connection to an electronic medical record (EMR) system with Wi-Fi, Ethernet, Bluetooth or Bluetooth Low Energy; ability to enter height, weight and body mass index (BMI); help identify signs of patient deterioration with automated Early Warning Scores (EWSs); customize workflows to document patient observations and vitals modifiers; and secure patient information with data encryption and additional security features. The patient monitoring device 200 can share aspects of the patient monitoring device described in U.S. Pat. No. 9,591,974B2, issued Mar. 14, 2017, entitled CONFIGURABLE HEALTH-CARE EQUIPMENT APPARATUS, the disclosure of which is herein incorporated by reference in its entirety.

In the example illustrated in FIG. 2, the patient monitoring device 200 includes a housing 202 having a display 204 that displays physiological parameters captured by one or more sensors that are integrated with or plugged into the housing 202 a such as a temperature sensor 206. The patient monitoring device 200 can include additional types of sensors such as a blood pressure sensor, a pulse oximetry sensor, and a respiration rate sensor. The display 204 is a touchscreen that receives tactile inputs from a user to enter information and to navigate between different tabs and/or pages within an application 208 displayed on the display 204.

The housing 202 can be mounted on a portable frame that has casters for allowing the patient monitoring device 200 to be moved around the healthcare facility such as from patient room to patient room. Alternatively, the patient monitoring device 200 can be stationary such that it can be mounted to a wall or attached to another fixture within the healthcare facility.

The application 208 uses data captured by the one or more sensors integrated with or plugged into the patient monitoring device 200 to generate one or more outputs. For example, the application 208 can display information on the display 204 based on the data captured by the one or more sensors such as one or more physiological variable measurements, one or more trends of the one or more physiological variable measurements over time, a directive or recommendation to perform a clinical intervention based on the physiological variable measurements, or other clinically relevant information based on the data captured by the one or more sensors.

As another example, the application 208 can calculate one or more scores based on the data captured by the one or more sensors. The application 208 can display the one or more scores in a user interface displayed on the display 204 of the patient monitoring device 200. The one or more scores can include customized scores relevant to patient risk and deterioration such as an early warning score, a sepsis risk score, a falls risk score, a pressure ulcer risk score, and/or other types of scores that are computed based on the data captured by the one or more sensors.

Additionally, the application 208 can trigger an alarm on the patient monitoring device 200 based on the data captured by the one or more sensors. The alarm can include a visual alarm such as a blinking light emitted by the display 204 or elsewhere on the patient monitoring device 200. The alarm can also include an audible alarm emitted by a speaker on the patient monitoring device 200, or elsewhere in a clinical environment where the patient monitoring device 200 is located. The application 208 can define one or more thresholds for triggering the alarm, and/or computation of a score for comparison to the one or more thresholds.

FIG. 3 illustrates an example of a communications device 300 that can be included in the third fleet of medical devices 106 in the system 10. In the example illustrated in FIG. 3, the communications device 300 is a smartphone. In alternative examples, the communications device 300 can include a tablet computer or a stationary workstation computer.

As shown in FIG. 3, the communications device 300 includes a display 302 that displays a user interface having a bibliographic section 304 that identifies a patient's name (e.g., “Hill, Larry”), medical record number (MRN) (e.g., “MRN: 176290”), date of birth (e.g., “DOB: 08/22/1943”), age (e.g., “Age: 76”), sex (e.g., “Male”), identified risks (e.g., falls risk, pneumonia risk, injury risk, etc.), and primary diagnosis (e.g., “pneumonia”).

The user interface displayed on the display 302 of the communications device 300 includes a vital signs dashboard 306 that displays physiological measurements such as non-invasive blood pressure (NIBP), SpO2, respiration rate, hear rate, temperature, and level of consciousness (LOC) (e.g., “lethargic”). At least some of the physiological measurements displayed in the vital signs dashboard 306 are captured by the one or more sensors of the patient monitoring device 200. The vital signs dashboard 306 further displays a modified early warning score (MEWS) that is calculated based on the physiological measurements. In the example illustrated in FIG. 3, the MEWs includes an arrow icon pointing upwards to indicate that the MEWS is trending up, and a time stamp to indicate the last time it was updated. Alternatively, the arrow icon can point downwards to indicate that the MEWS is trending down.

The user interface displayed on the display 302 of the communications device 300 further includes a care communication section 310 having selectable icons that when selected display additional information related to the care of the patient. The selectable icons include icons that when selected display caregivers assigned to the patient, the patient's lab results, reminders on care protocols for the patient, and alerts generated for the patient.

The user interface displayed on the display 302 of the communications device 300 further includes an output section 308 generated by the application 208 when installed on the communications device 300. One or more outputs generated by the application 208 can be displayed in the output section 308 such as information based on the data captured by the one or more sensors on the patient monitoring device 200. For example, the output section 308 can include one or more physiological variable measurements, one or more trends of the one or more physiological variable measurements over time, a directive or recommendation to perform a clinical intervention, or other clinically relevant information based on the data captured by the one or more sensors. The one or more outputs generated by the application 208 for display in the output section 308 can further include one or more scores calculated based on the data captured by the one or more sensors, and/or one or more alarms that are triggered based on the data captured by the one or more sensors of the patient monitoring device 200.

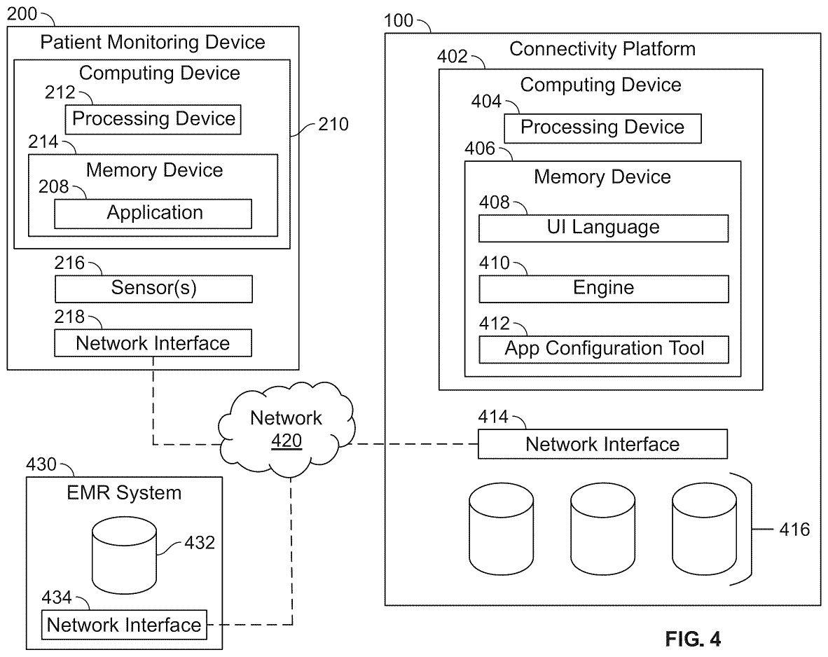

FIG. 4 schematically illustrates an example of the connectivity platform 100 communicatively coupled over a network 420 to the patient monitoring device 200. As shown in the illustrative example of FIG. 4, the connectivity platform 100 includes a computing device 402 having a processing device 404 and a memory device 406. The processing device 404 is an example of a processing unit such as a central processing unit (CPU). The processing device 404 can include one or more central processing units (CPUs). In some examples, the processing device 404 is part of a processing circuitry that can include one or more digital signal processors, field-programmable gate arrays, and/or other types of electronic circuits.

The memory device 406 stores data and instructions for execution by the processing device 404. The memory device 406 stores a user interface language specification 408, an engine 410, and an application configuration tool 412 for updating the configurable settings of the application 208 installed on the medical devices in the system 10 such as the hospital beds, the patient monitoring devices 200, and the communications device 300.

As will be described in more detail below, the user interface language specification 408 defines an application configuration registration (ACR), and the engine 410 identifies and processes a custom resource definition of the ACR to dynamically render a user interface in a configuration portal provided by the application configuration tool 412. The user interface in the configuration portal can be used to adjust one or more configurable settings of the application 208 installed on the patient monitoring device 200. The user interface language specification 408, the engine 410, and the application configuration tool 412 allow multiple applications to be configured in a single configuration portal without having to update the configuration portal to accommodate changes to the configurable settings of the applications.

The ACR is different from an application programming interface (API) in that the ACR enables dynamic declaration and rendering of a user interface that collects, persists, and disseminates (or otherwise makes available) all configuration data that is required to support a specific deployment of a given application in a generic manner. At least one benefit of the ACR is that it enables configuration functionality for any given application built for execution on one or more of the plurality of medical devices. The ACR is an examples of a software engine that makes use of APIs. In some instances, the ACR is referred to herein as a user interface engine.

The memory device 406 includes computer-readable media, which may include any media that can be accessed by the processing device 404. The computer-readable media includes non-transitory computer-readable storage media and computer-readable communication media.

The computer-readable storage media includes volatile and nonvolatile, removable and non-removable media implemented in any device configured to store information such as computer-readable instructions, data structures, program modules, or other data. The computer-readable storage media can include, but is not limited to, random access memory, read only memory, erasable programmable read-only memory (EPROM), flash memory, and other memory technology, including any medium that can be used to store information that can be accessed by the processing device 404. The computer-readable storage media is non-transitory.

The computer-readable communication media embodies computer-readable instructions, data structures, program modules, or other data in a modulated data signal such as a carrier wave or other transport mechanism and includes any type of delivery media. The term “modulated data signal” refers to a signal that has one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are within the scope of computer-readable media.

The connectivity platform 100 includes a network interface 414 to connect the connectivity platform 100 to the network 420. The network interface 414 can include a wired interface such as an ethernet cable port to connect the connectivity platform 100 to the network 420 and/or wireless interfaces to wirelessly connect the connectivity platform 100 to the network 420 such as through Wi-Fi, ultra-wideband (UWB), and other wireless connections.

The connectivity platform 100 communicate over the network 420 with the medical devices of the system 10 such as the hospital beds, the patient monitoring devices 200, and the communications devices 300. Also, the connection to the network 420 allows the connectivity platform 100 to communicate with an electronic medical record system (EMR) 430 that includes a database 432 for housing a plurality of electronic medical records (EMRs) each associated with a patient of the healthcare facility. The EMR system 430 can include a network interface 434 to facilitate connection to the network 420, as is shown in the example of FIG. 4.

As further shown in FIG. 4, the patient monitoring device 200 is schematically illustrated as including a network interface 218 that can be used to connect the patient monitoring device 200 to the network 420 via a wired connection, a wireless connection, or any combination thereof. The patient monitoring device 200 includes a computing device 210 having a processing device 212 and a memory device 214 that can have similar aspects as the computing device 402, the processing device 404, and the memory device 406 of the connectivity platform 100. As shown in FIG. 4, the memory device 214 stores the application 208. The patient monitoring device 200 is further schematically illustrated as including one or more sensors 216 such as a temperature sensor, a blood pressure sensor, a pulse oximetry sensor, a respiration rate sensor, or other sensor that can be used to capture physiological variable measurements.

The connectivity platform 100 includes one or more databases 416 that are utilized by the application 208 installed on the patient monitoring device 200. The connectivity platform 100 utilizes the one or more databases 416 to persist data collected by the sensors of the medical devices in the system 10 such as the one or more sensors 216 of the patient monitoring device 200. Prior to persisting the data in the one or more databases 416, the connectivity platform 100 processes the data such as to translate the data into a standard representation that is recognized by the application 208 installed on the patient monitoring device 200.

Once the sensor data is translated and persisted into the one or more databases 416 by the connectivity platform 100, the data is made available to the applications that are installed on the medical devices in the system 10. For example, the data is made available to the application 208 installed on the patient monitoring device 200 for generating one or more outputs.

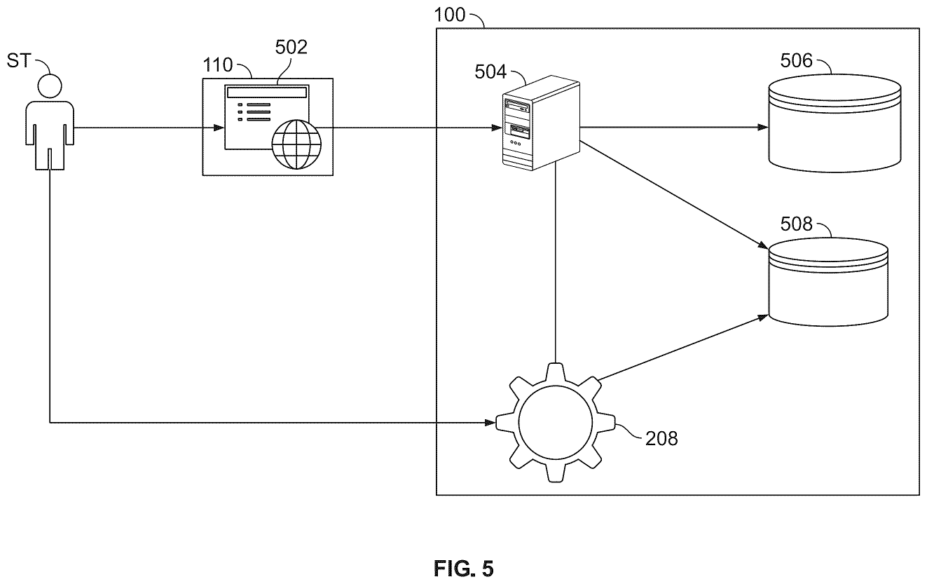

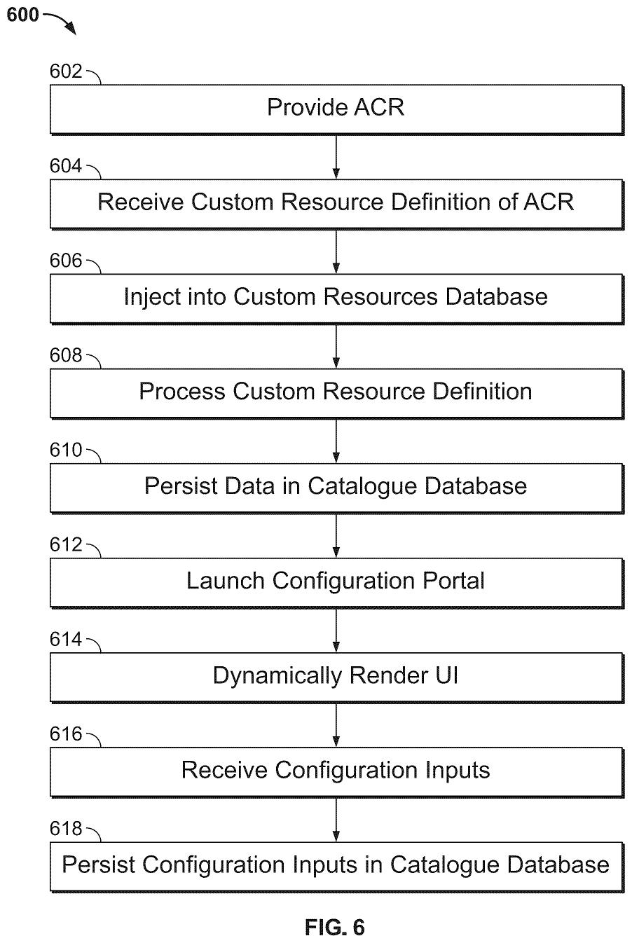

As an illustrative example, the application 208 uses the data persisted in the one or more databases 416 to generate one or more outputs on the patient monitoring device 200 such as to display one or more physiological variable measurements or one or more trends of the one or more physiological variable measurements over time, a directive or recommendation to perform a clinical intervention based on the physiological variable measurements, or other clinically relevant information. As another illustrative example, the application 208 can use the data persisted in the one or more databases 416 to calculate one or more scores relevant to patient risk and/or deterioration such as an early warning score, a sepsis risk score, a falls risk score, a pressure ulcer risk score, and other risk scores. Additionally, the application 208 can use the data persisted in the one or more databases 416 of the connectivity platform 100 to trigger one or more alarms or alerts. The one or more databases 416 include additional databases for facilitating execution of the application 208 as will be described with reference to FIGS. 5 and 6.

FIG. 5 schematically illustrates additional aspects of the connectivity platform 100. In this example, the connectivity platform 100 includes a configuration portal 502 and a catalog service 504 that recognizes and processes a custom resource definition of the ACR constructed for the application 208. The catalog service 504 persists the custom resource definition into a catalog service database 506. The connectivity platform 100 further includes a custom resources database 508. The interoperation of the configuration portal 502, the catalog service 504, the catalog service database 506, and the custom resources database 508 causes a user interface to be dynamically rendered for selection of configuration inputs to configure the application 208 in the configuration portal 502 displayed on the display monitor 110 of the workstation computer 108.

A custom resource definition is defined by the catalog service 504. The custom resource definition is an implementation of the ACR. The custom resource definition is read by the configuration portal 502 using the engine 410. The custom resource definition once created for the application 208 can be updated at any time to modify configuration elements specific the application 208 for adjusting one or more configurable settings of the application 208.

The interaction between the catalog service 504, the configuration portal 502, and the application 208 allows updates to the configuration elements displayed on the user interface within the configuration portal 502 outside of a new release of the connectivity platform 100.

FIG. 6 schematically illustrates an example of a method 600 of managing medical device applications that can be performed by the connectivity platform 100. Referring now to FIGS. 5 and 6, the method 600 includes an operation 602 of providing an application configuration registration (ACR). The ACR is owned and controlled by the connectivity platform 100. The ACR acts as an interface specification between applications configured for installation on the medical devices and the connectivity platform 100 for the purposes of obtaining application specific configuration data. The interface specification provided by the ACR includes the user interface language specification 408 that is discussed above.

The ACR represents a complete set of dynamically configurable data associated with the application 208 that is maintained by the catalog service 504. The ACR can be used to define a connection type within the configuration portal 502 for configuring the application 208 on an entity-by-entity basis. The connection type defines the user interface that enables collection of all configurable data associated with a given connection type within the configuration portal 502 when configuring an instance of the given connection type. The connection type further defines how and where the configurable data associated with an instance of a given connection type is persisted within the catalog service database 506. The connection type defines the configuration of other software components such as an enterprise gateway when an instance of a connection type is configured. The connection type further defines how an instance of a given connection type is displayed on the configuration portal 502. As an illustrative example, custom resource definitions of the ACR can be processed every minute, and this interval is configurable.

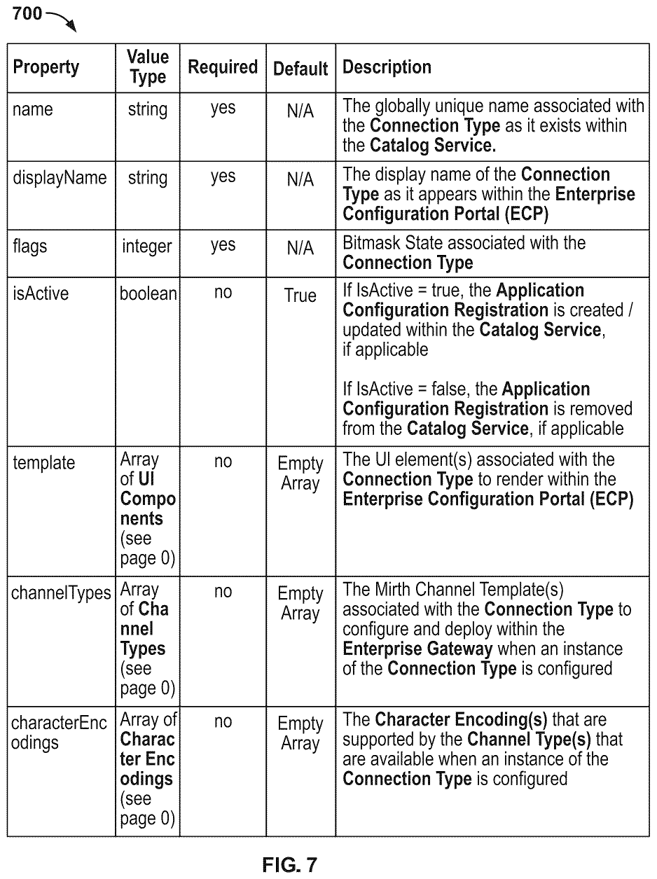

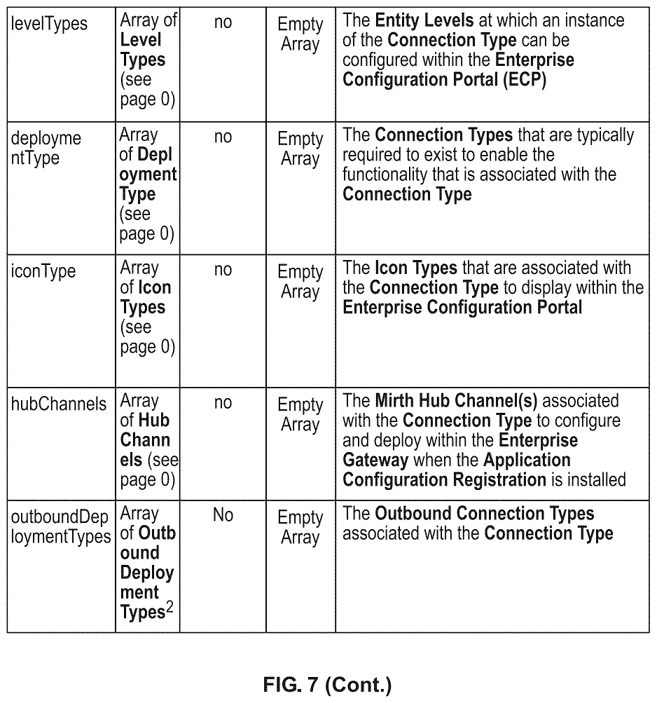

FIGS. 7-37 provide examples of the user interface language specification 408 that can be used to construct custom resource definitions of the ACR by the connectivity platform 100. The user interface language specification 408 is processed by the engine 410 to generate a user interface within the configuration portal 502 for adjusting one or more configurable settings of the application 208 without having to update or modify to the connectivity platform 100 itself. FIGS. 7-37 provide a plurality of tables that describe the options available to applications that are built for execution on one or more of the plurality of medical devices that wish to leverage the ACR functionality. Thus, the tables demonstrate the dynamic nature of the ACR functionality.

FIG. 7 shows a table 700 providing an example of a root of the ACR custom resource definition that can be stored in the catalog service database 506 for defining overarching properties and structures associated with the connection type for the application 208.

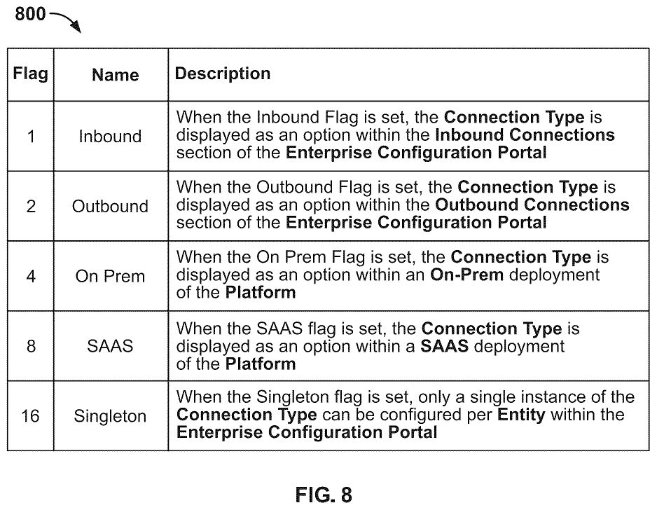

FIG. 8 shows a table 800 providing an example of bitmask flags associated with a given connection type that define when, where, and how an instance of a given connection type can be configured within the configuration portal 502.

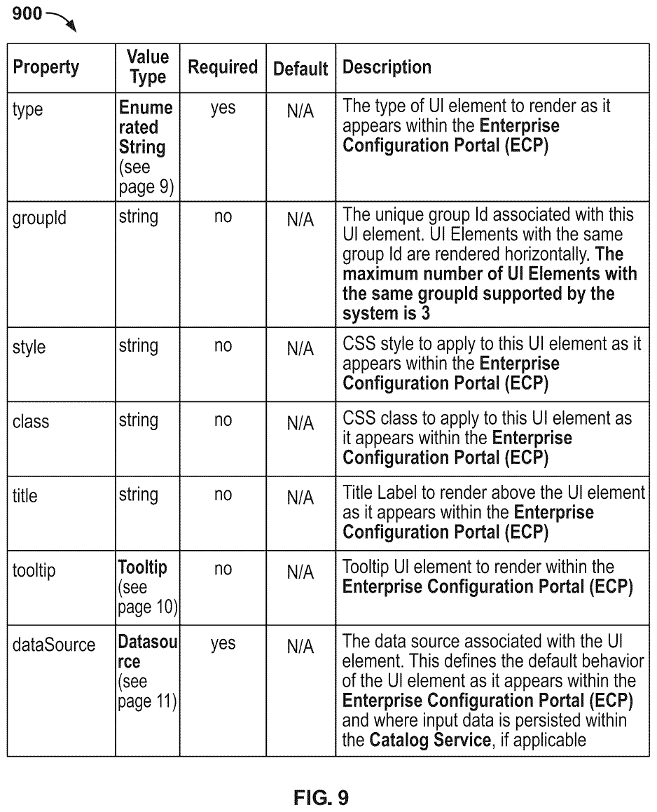

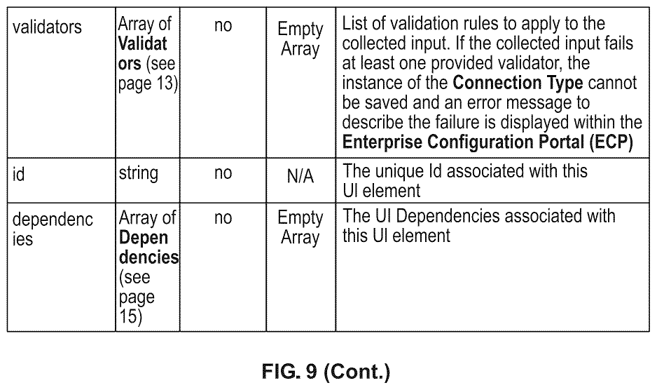

FIG. 9 shows a table 900 providing an example of a user interface element associated with the connection type to render within the configuration portal 502. A given connection type can define any number of user interface components to meet business logic requirements.

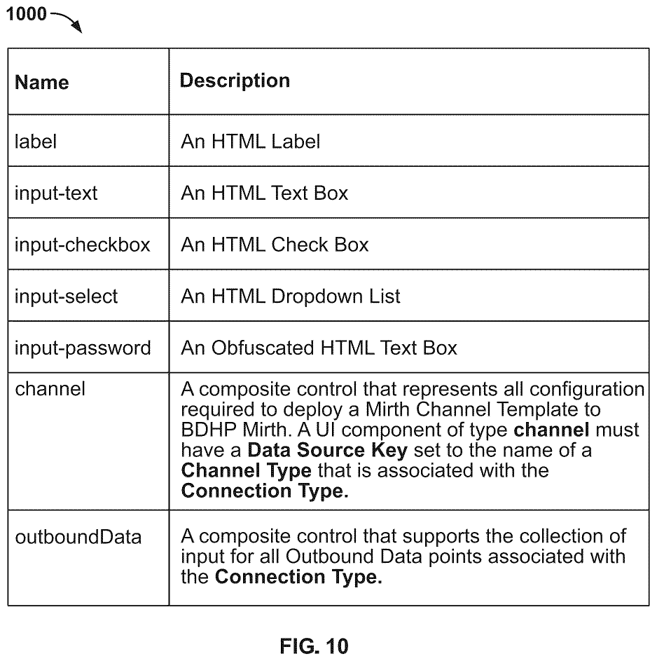

FIG. 10 shows a table 1000 providing an example of a type of user interface element to render as it appears within the configuration portal 502.

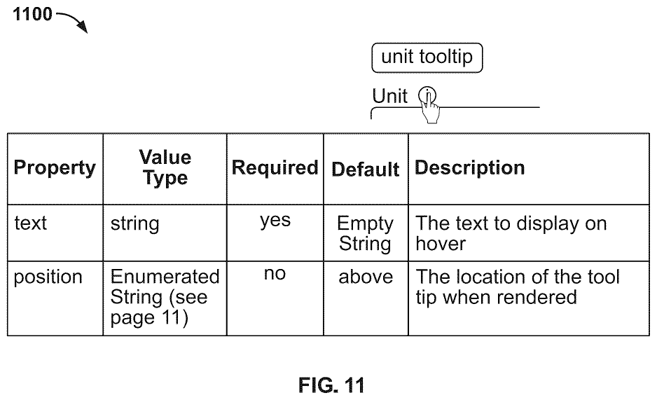

FIG. 11 shows a table 1100 providing an example of tooltip user interface elements to render within the configuration portal 502 associated with a user interface component. A tooltip user interface element is rendered as a blue encircled icon. When the user hovers over the icon, the defined text is displayed in the defined position.

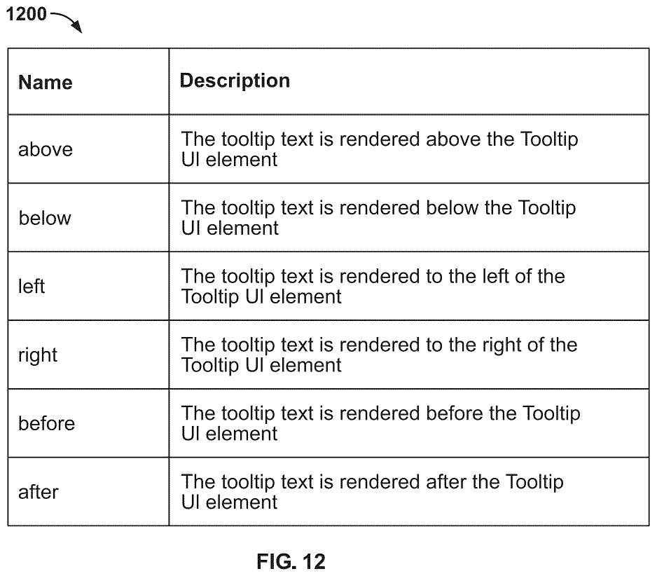

FIG. 12 shows a table 1200 providing locations of the tool tip when rendered.

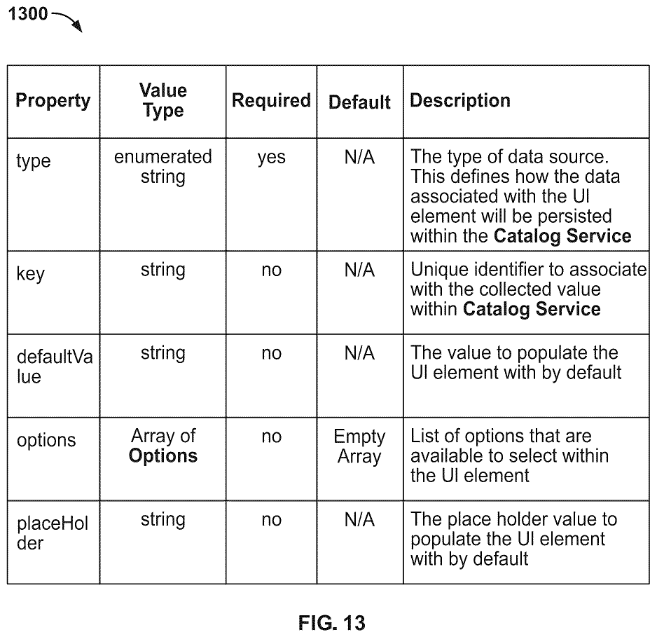

FIG. 13 shows a table 1300 providing examples of data sources associated with the user interface element. This defines the default behavior of the user interface element as it appears within the configuration portal 502 and where input data is persisted within the catalog service database 506, if applicable.

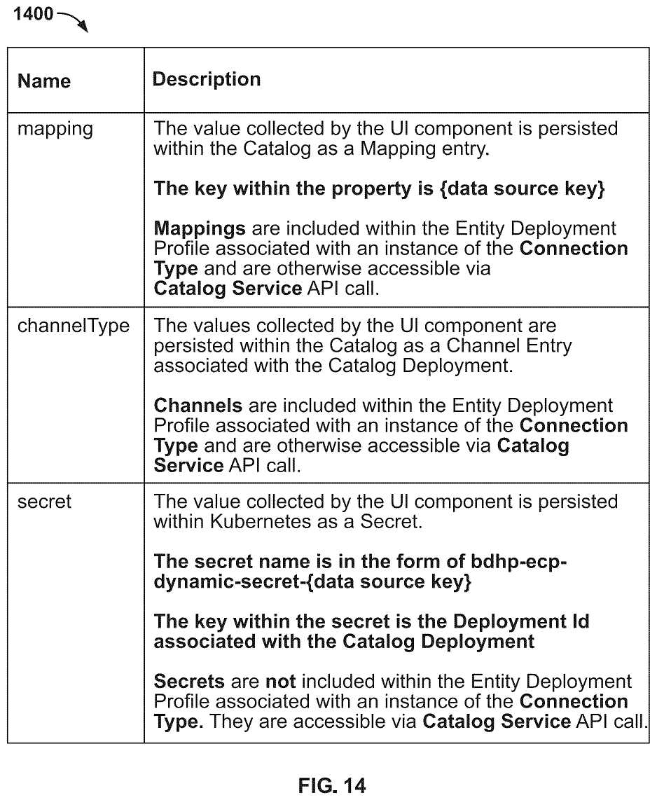

FIG. 14 shows a table 1400 providing an example of types of data sources that define how the data associated with the user interface element will be persisted within the catalog service database 506.

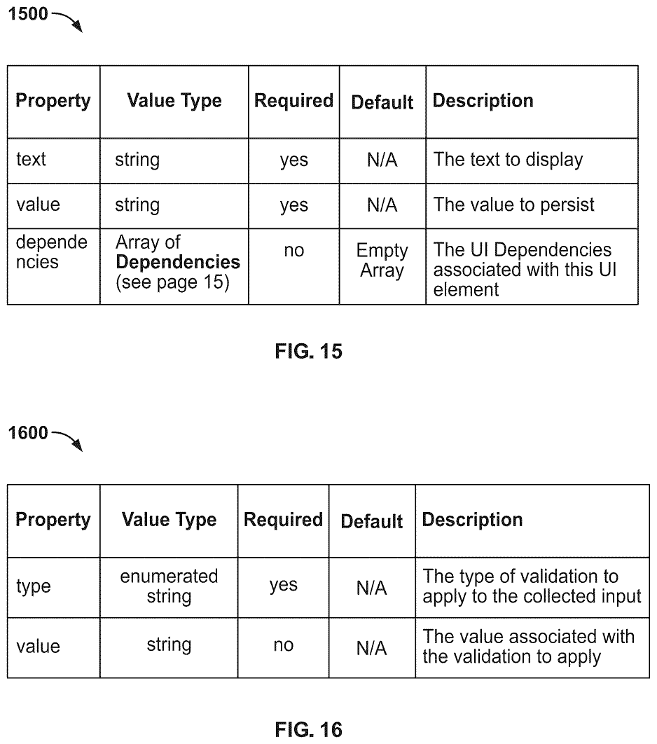

FIG. 15 shows a table 1500 providing examples of options associated with the user interface component. A given user interface component can define any number of options to meet business logic requirements for the configuration portal 502.

FIG. 16 shows a table 1600 providing examples of validation rules to apply to the collected user interface input. If the collected user interface input fails at least one of the provided validators, the instance of the connection type cannot be saved and an error message to describe the failure is displayed within the configuration portal 502.

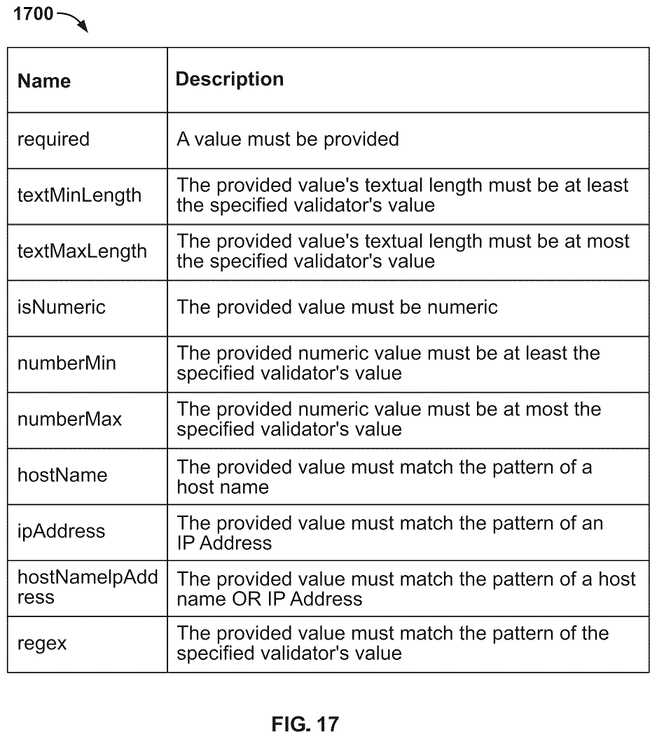

FIG. 17 shows a table 1700 providing examples of the types of validations to apply to the collected user interface input received from the configuration portal 502.

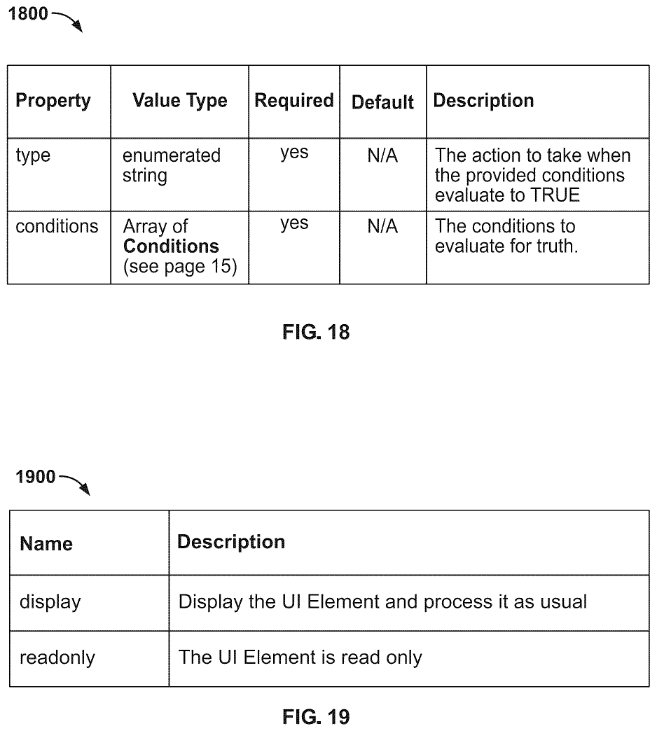

FIG. 18 shows a table 1800 providing examples of dependencies associated with the user interface element displayed in the configuration portal 502.

FIG. 19 shows a table 1900 providing examples of actions to take when the provided conditions evaluate to TRUE in the configuration portal 502.

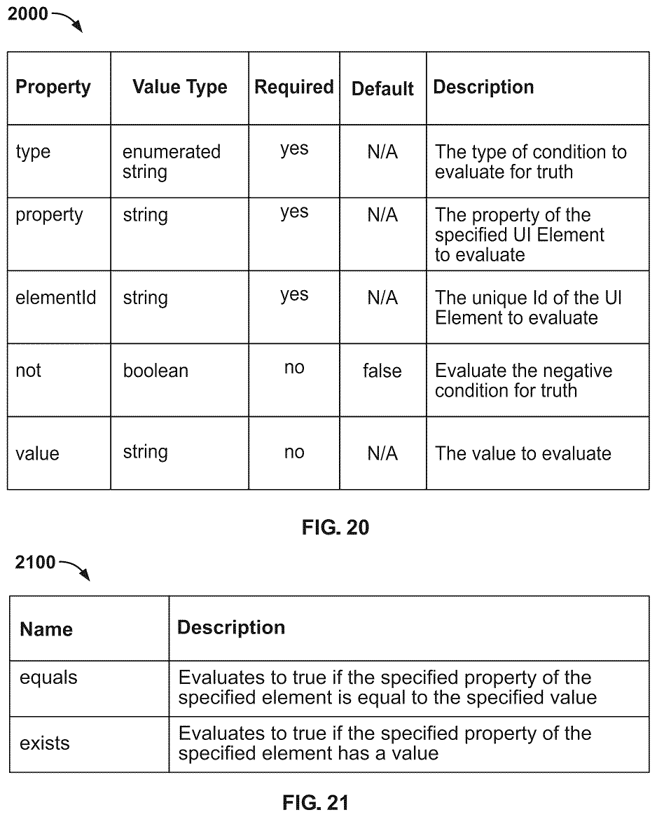

FIG. 20 shows a table 2000 providing examples of condition to evaluate in the configuration portal 502.

FIG. 21 shows a table 2100 providing examples of types of conditions to evaluate for truth in the configuration portal 502.

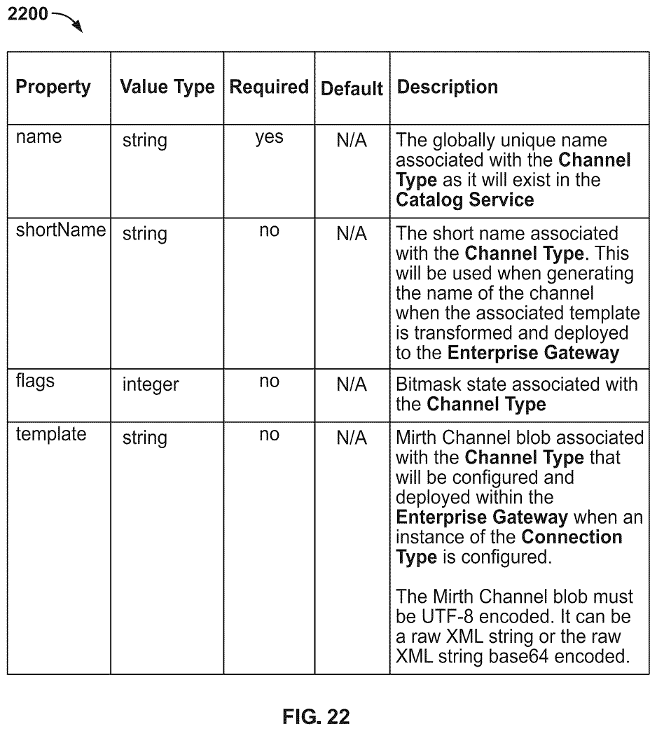

FIG. 22 shows a table 2200 providing examples of mirth channel template(s) associated with the connection type to configure and deploy within the enterprise gateway when an instance of the connection type is configured using the configuration portal 502.

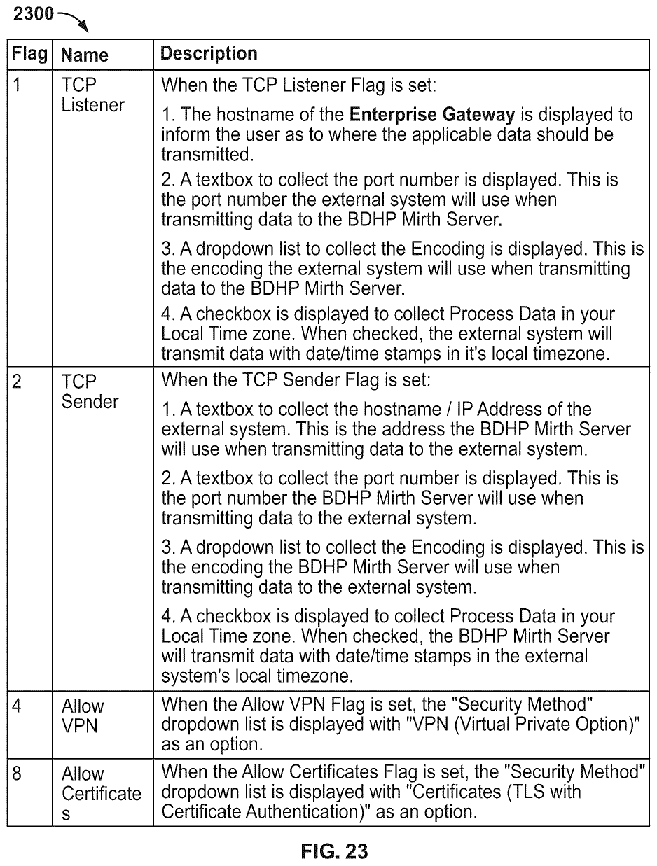

FIG. 23 shows a table 2300 providing examples of bitmask flags associated with a given channel type that define when, where, and how an instance of the given channel type can be configured within the configuration portal 502.

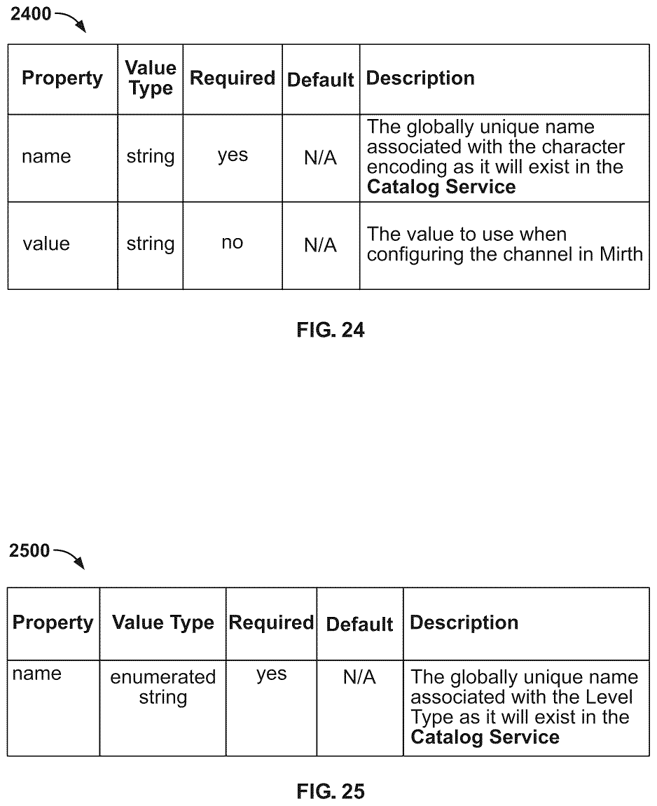

FIG. 24 shows a table 2400 providing examples of character encodings that are supported by the channel types that are available when an instance of the connection type is configured within the configuration portal 502.

FIG. 25 shows a table 2500 providing examples of entity levels at which an instance of the connection type can be configured within the configuration portal 502.

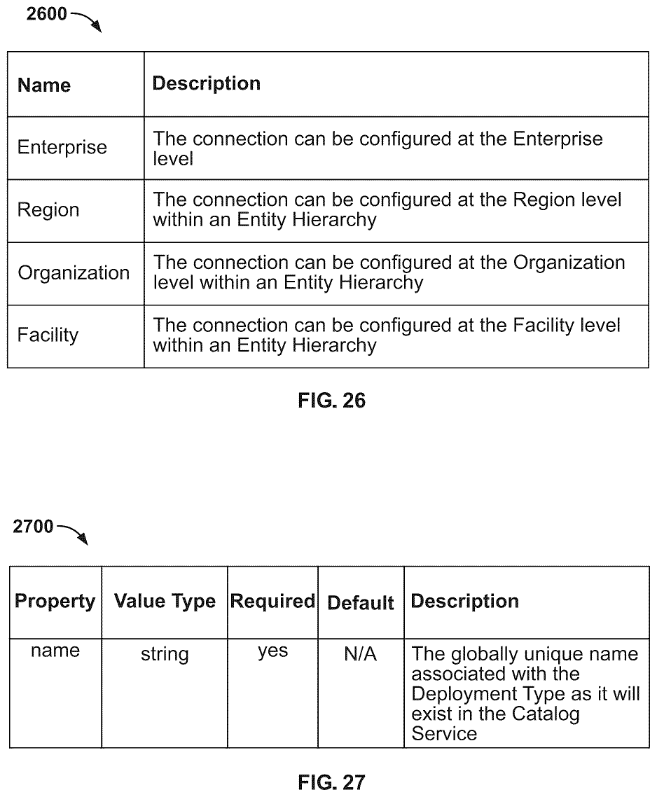

FIG. 26 shows a table 2600 providing examples of globally unique names associated with the level types that exist in the catalog service 504.

FIG. 27 shows a table 2700 providing an example of a deployment type that can be required to exist to enable the functionality that is associated with the connection type. If an instance of a given connection type is configured at a given entity, and the entity is not covered by a defined deployment type, a warning is displayed to the user.

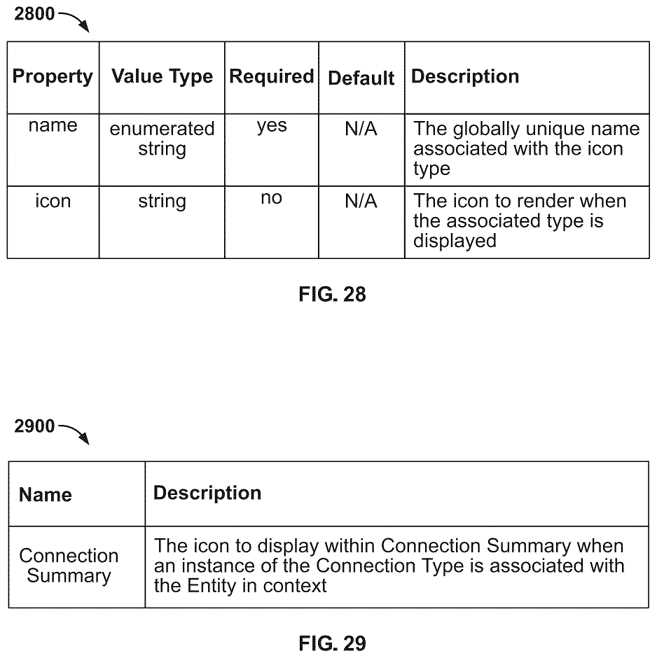

FIG. 28 shows a table 2800 providing examples of icon types that are associated with the connection type to display within the configuration portal 502.

FIG. 29 shows a table 2900 providing an example of globally unique name associated with the icon types shown in the table 2800 of FIG. 28.

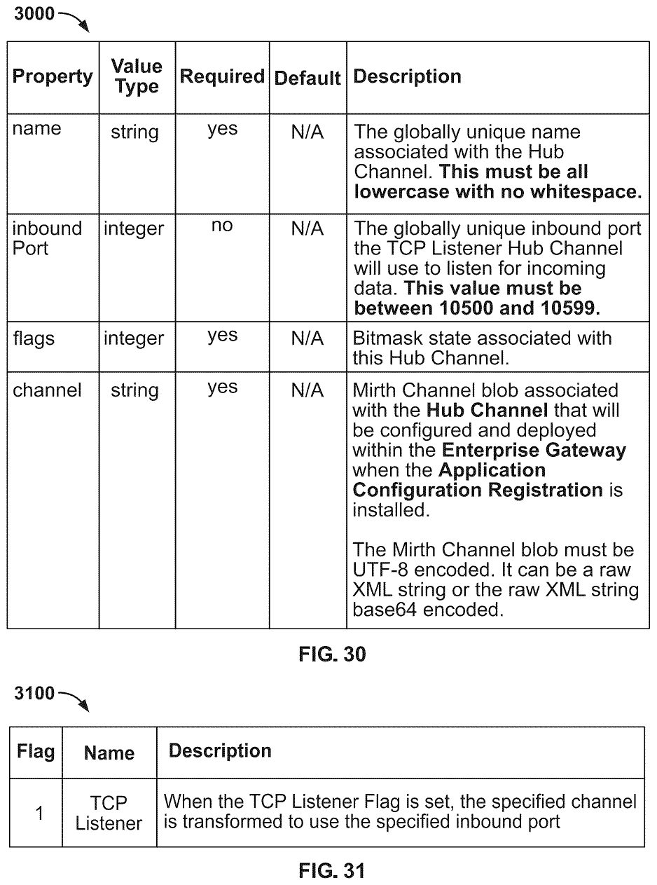

FIG. 30 shows a table 3000 providing examples of hub channels associated with the connection type to configure and deploy within the enterprise gateway when the ACR custom resource definition is installed on the connectivity platform 100.

FIG. 31 shows a table 3100 providing examples of hub bitmask flags associated with a given hub channel that define when, where, and how the specified channel is transformed and deployed to the enterprise gateway by the connectivity platform 100.

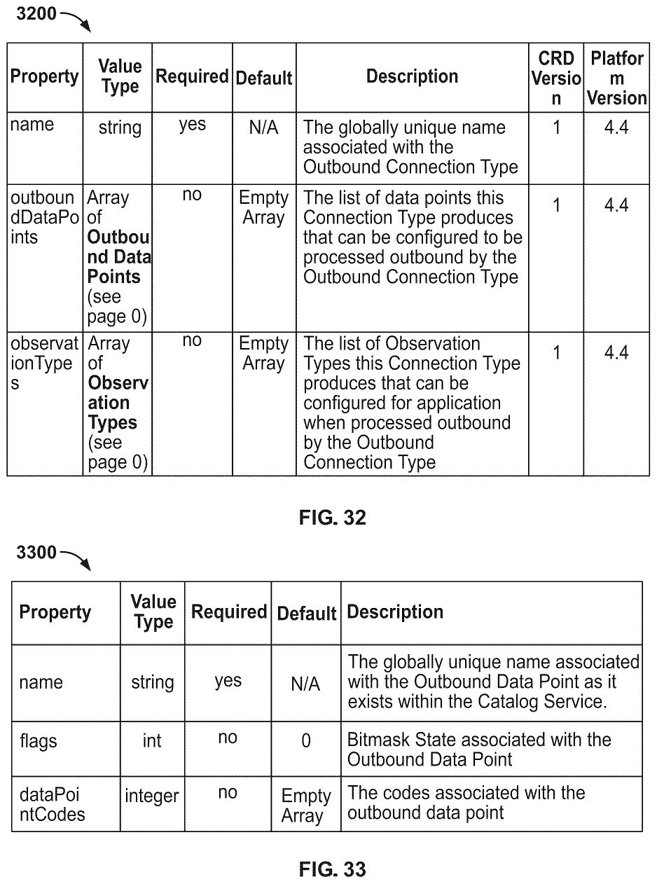

FIG. 32 shows a table 3200 providing examples of outbound connection types associated with the connection type within the configuration portal 502.

FIG. 33 shows a table 3300 providing examples of outbound data points that the connection type produces that can be configured to be processed outbound by the outbound connection type within the configuration portal 502.

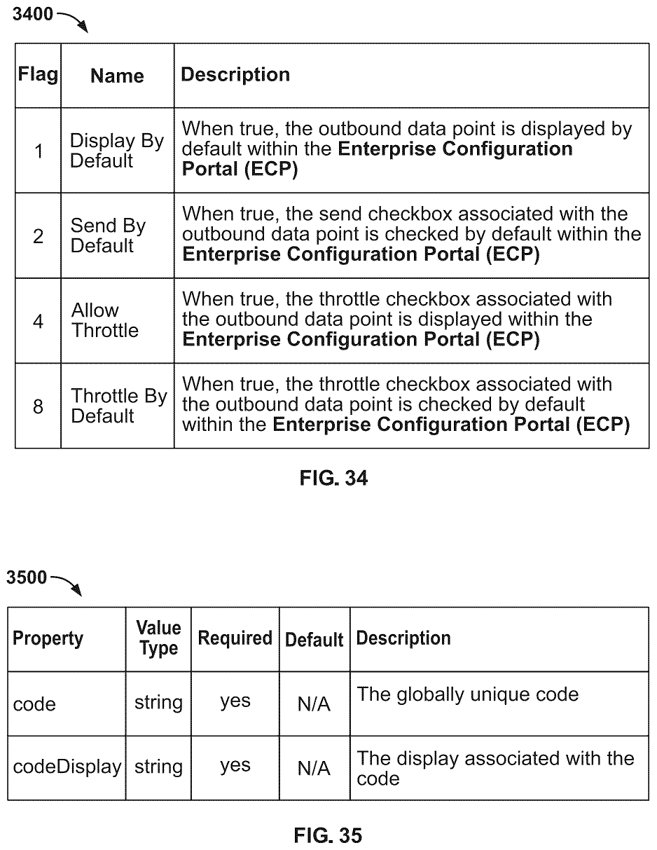

FIG. 34 shows a table 3400 providing examples of bitmask states associated with the outbound data points in the table 3300 of FIG. 33.

FIG. 35 shows a table 3500 providing examples of data point codes associated with the outbound data point within the configuration portal 502.

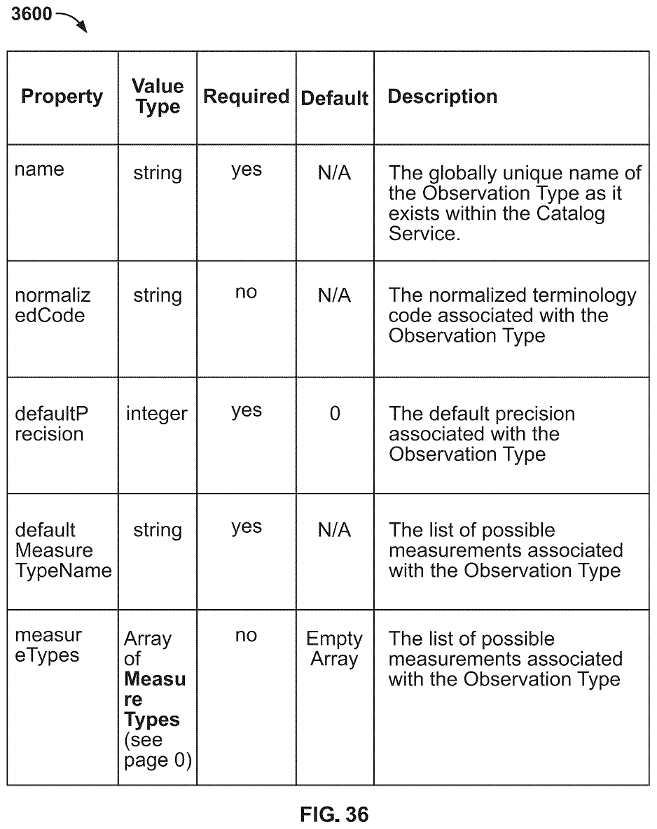

FIG. 36 shows a table 3600 providing examples of observation types that the connection type produces that can be configured for the application 208 when processed outbound by the outbound connection type.

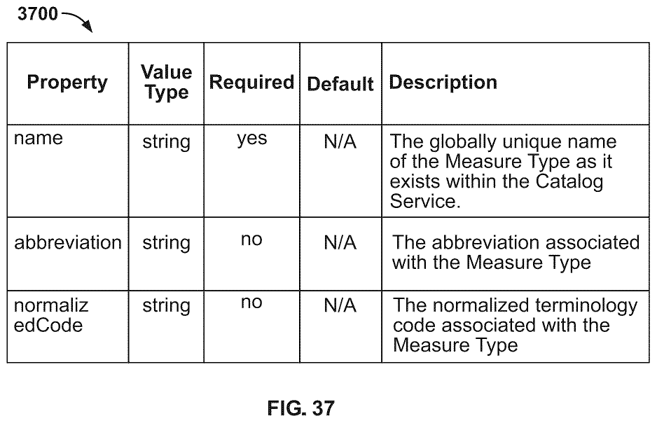

FIG. 37 shows a table 3700 providing examples of possible measurements associated with the observation types in the table 3600 of FIG. 36.

Referring back to FIG. 6, the method 600 includes an operation 604 of receiving a custom resource definition of the ACR that is constructed for the application 208. As described above, the application 208 is configured for execution on a medical device such as on the patient monitoring device 200. The custom resource definition of the ACR defines the connection type within the configuration portal 502 for the application 208.

The method 600 includes an operation 606 of injecting the custom resource definition into the custom resources database 508. The custom resources database 508 is maintained by a container orchestration system for automating software deployment, scaling, and management.

The method 600 includes an operation 608 of processing the custom resource definition of the ACR. Operation 608 can include using the catalog service 504 to recognize that the custom resource definition of the ACR has been created or updated, process the custom resource definition of the ACR, and persist all data associated with the custom resource definition of the ACR within the catalog service database 506.

After completion of operation 608, the application 208 is configurable within the configuration portal 502. When the service technician ST launches the configuration portal 502 and selects the application 208 within the configuration portal 502, the configuration portal 502 obtains the ACR custom resource definition data defined for the application 208 from the catalog service 504, and dynamically renders a user interface based upon the user interface language specification 408 provided by the ACR custom resource definition of the application 208. As described above, the user interface language specification 408 defines what user interface elements should be rendered to a screen displayed within the configuration portal 502 for the application 208, how the user interface elements should be rendered to the screen, and how data collected within the user interface elements should be persisted and subsequently made available to the application 208 when installed on one or more of the medical devices of the system 10.

The method 600 includes an operation 612 of launching the configuration portal 502. The connectivity platform 100 displays the configuration portal 502 on the display monitor 110 of the workstation computer 108. The configuration portal 502 is launched in response to an input from the service technician ST on the workstation computer 108 such as when the service technician ST requests access to the connectivity platform 100 to adjust one or more configurable settings of the application 208 after installation on one or more medical devices.

The method 600 includes an operation 614 of dynamically rendering a user interface in the configuration portal 502 based on the custom resource definition of the ACR. For example, when the service technician ST launches the configuration portal 502 and selects the application 208, the configuration portal 502 obtains the custom resource definition data of the application 208 from the catalog service 504 for dynamically rendering the user interface.

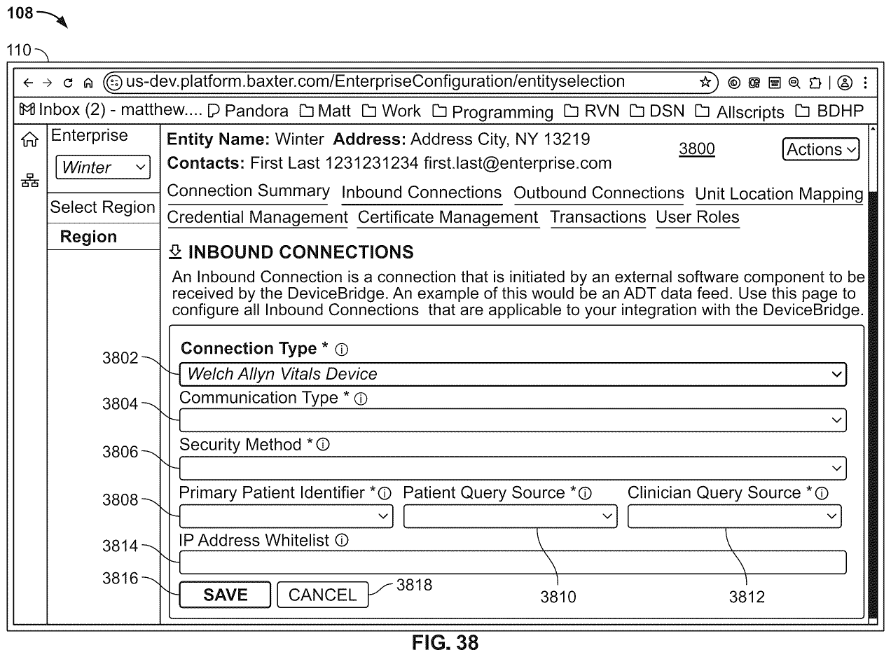

FIG. 38 illustrates an example of a user interface 3800 that can be dynamically rendered in the configuration portal 502 during operation 614 of the method 600. As shown in FIG. 38, the user interface 3800 includes a plurality of data fields for entering configuration inputs for an inbound connection type for the application 208. For example, the plurality of data fields can include a data field 3802 for defining an inbound connection type, a data field 3804 for defining a communication type, a data field 3806 for defining a security method, a data field 3808 for defining a primary patient identifier, a data field 3810 for defining a patient query source, a data field 3812 for defining a clinician query source, and a data field 3814 for defining an internet protocol (IP) address whitelist. At least some of the data fields can include drop-down menus (e.g., the data fields 3802-3812) for selecting a configuration input. Some of the data fields may have a space for typing text (e.g., the data field 3814). FIG. 38 is provided by way of illustrative example such that the number, type, and configuration of data fields may vary.

The data fields displayed in the user interface 3800 are configurable based on the custom resource definition of the ACR. The custom resource definition can be modified to display different types of data fields for entering different types of configuration inputs for the inbound connection type for the application 208. In examples where the data fields have drop-down menus, the custom resource definition can be modified to include different options for selection in the drop-down menus. The modifications to the custom resource definition dynamically render the user interface 3800 without requiring any updates to the connectivity platform 100 such that modifying the options for configurating the application 208 is accomplished independently and asynchronously with respect to the connectivity platform 100.

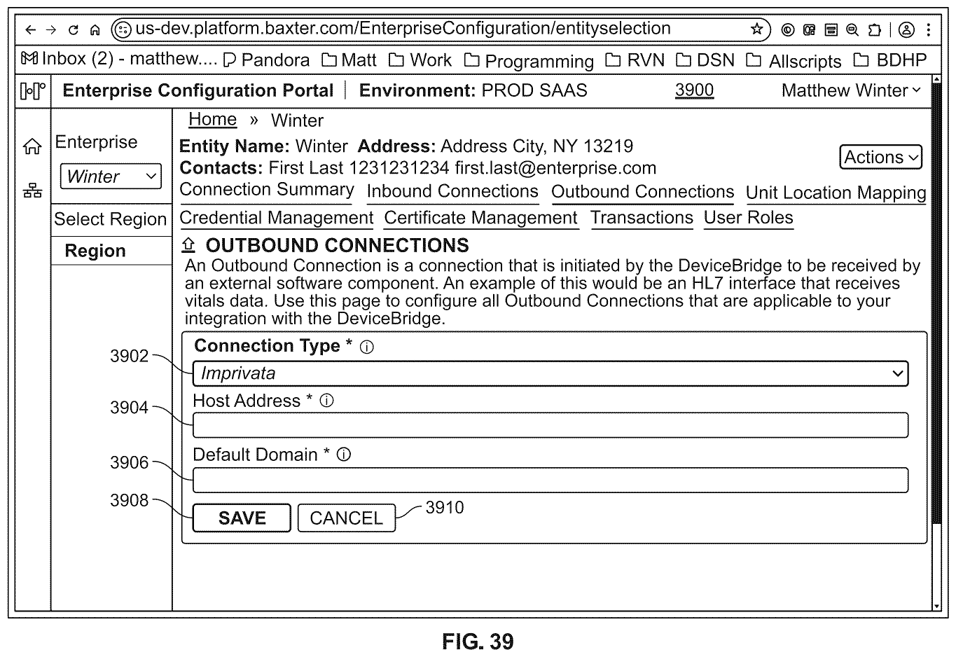

FIG. 39 illustrates another example of a user interface 3900 that can be dynamically rendered in the configuration portal 502 during operation 614 of the method 600. As shown in FIG. 39, the user interface 3900 includes a plurality of data fields for receiving configuration inputs for an outbound connection type for the application 208. The plurality of data fields can include a data field 3902 for defining an outbound connection type, a data field 3904 for defining a host address, and a data field 3906 for defining a default domain.

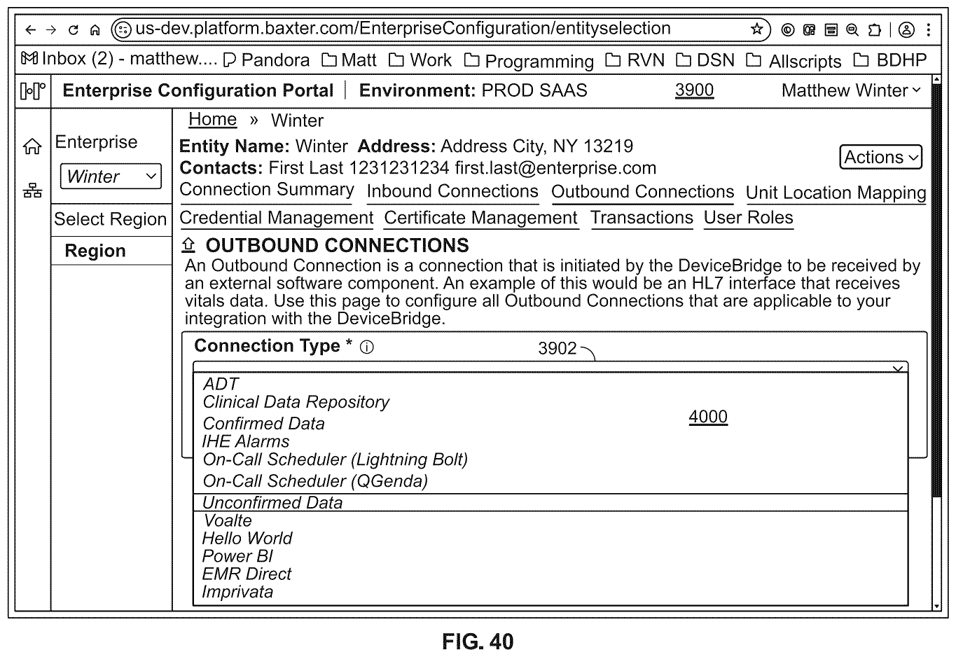

At least some of the data fields can include drop-down menus for selecting a configuration input. For example, FIG. 40 illustrates an example of a drop down menu 4000 for defining the outbound connection type in the data field 3902 shown in FIG. 39.

Some of the other data fields in the user interface 3900 provide space for typing text (e.g., the data fields 3904 and 3906). FIGS. 39 and 40 are provided by way of illustrative example such that it is contemplated that the number, type, and configuration of data fields may vary for the outbound connection type. The data fields displayed in the user interface 3900 are configurable based on the custom resource definition of the ACR.

The method 600 includes an operation 616 of receiving configuration inputs on the user interface rendered in operation 614. The configuration inputs are received when the service technician ST enters one or more configuration inputs in one or more data fields of the dynamically rendered user interface displayed within the configuration portal 502 and selects a save icon (e.g., save icon 3816 on the user interface 3800 of FIG. 38 and save icon 3908 on the user interface 3900 of FIG. 39). Alternatively, when the service technician ST selects a cancel icon (e.g., cancel icon 3818 on the user interface 3800 of FIG. 38 and cancel icon 3910 on the user interface 3900 of FIG. 39), the configuration inputs entered into the one or more data fields are discarded by the connectivity platform 100.

The method 600 includes an operation 618 of persisting the configuration inputs received in operation 616 into the catalog service database 506. For example, when the service technician provides the configuration inputs to the dynamically rendered user interface displayed in the configuration portal 502 and clicks save, the configuration data is persisted to the catalog service database 506 via the catalog service 504. At this point, the specified configuration data is available in the configuration portal 502 for the application 208 via the catalog service 504.

Following completion of operation 618, the application 208 when executed on the medical device is executed based on the configuration data persisted to the catalog service database 506. As described in the examples above, the application 208 can be executed on one or more of the medical devices in the system 10 such as the hospital beds, the patient monitoring device 200, the communications device 300, as well as other types of medical devices.

The application 208 when executed on the medical devices generates one or more outputs such as to display one or more physiological variable measurements or one or more trends of the one or more physiological variable measurements, a directive or recommendation to perform a clinical intervention based on the physiological variable measurements, or other clinically relevant information. As another illustrative example, the application 208 can use the data persisted in the catalog service database 506 to calculate one or more scores relevant to patient risk and/or deterioration such as an early warning score, a sepsis risk score, a falls risk score, a pressure ulcer risk score, and other risk scores. Also, the application 208 can use the data persisted in the catalog service database 506 to trigger one or more alarms or alerts.

In view of the foregoing, a technical advantage of the method 600 is allowing applications to describe and change their necessary configuration fields without having to update the connectivity platform 100, which operates the configuration portal 502. A further technical advantage is that the connectivity platform 100 provides a centralized location for the configuration data of the application 208 including the collection of the configuration data and the procurement of the configuration data by the application 208. This allows the connectivity platform 100 and the application 208 to operate asynchronously from each other.

A further technical advantage is that the ACR custom resource definition of the application 208 allows for any number of configurations to be established and managed for consumption by the application 208 within the connectivity platform 100. Another technical advantage includes the ability to update the ACR custom resource definition at any time. An updated ACR custom resource definition can be consumed and made available for adjusting one or more configurable settings of the application 208 as soon as the updated ACR custom resource definition is processed by the catalog service 504 of the connectivity platform 100.

The various embodiments described above are provided by way of illustration only and should not be construed to be limiting in any way. Various modifications can be made to the embodiments described above without departing from the true spirit and scope of the disclosure.

What is claimed is:

1. A system for managing medical device applications, the system comprising:
at least one processing device; and
a memory device storing instructions which, when executed by the at least one processing device, cause the at least one processing device to:
provide a user interface engine;
receive a custom resource definition of the user interface engine for an application configured for execution on a medical device;
process the custom resource definition;
launch a configuration portal;
dynamically render a user interface in the configuration portal, the user interface being rendered based on the custom resource definition;
receive configuration inputs on the user interface; and
persist the configuration inputs into a catalogue database that causes the application to be executed on the medical device based on the configuration inputs.

2. The system of claim 1, wherein the user interface engine provides an interface specification between the application and the configuration portal for obtaining configuration data for the application.

3. The system of claim 1, wherein the application and the configuration portal operate asynchronously with respect to one another.

4. The system of claim 1, wherein the memory device stores additional instructions which, when executed by the at least one processing device, cause the at least one processing device to:
receive one or more changes to the custom resource definition; and
update the user interface displayed in the configuration portal based on the one or more changes to the custom resource definition, wherein the user interface is updated without modifying the configuration portal.

5. The system of claim 1, wherein the user interface rendered in the configuration portal enables a plurality of configurations for execution of the application.

6. The system of claim 1, wherein the configuration inputs define at least one inbound connection and at least one outbound connection for the application.

7. The system of claim 1, further comprising:
the medical device, and wherein the medical device includes a sensor for capturing physiological data from a patient.

8. The system of claim 7, wherein the configuration inputs determine how the physiological data captured by the sensor of the medical device is persisted in a database.

9. The system of claim 7, wherein the medical device is a patient monitoring device or a hospital bed.

10. A method of managing medical device applications, the method comprising:
providing a user interface engine;
receiving a custom resource definition of the user interface engine for an application configured for execution on a medical device;
processing the custom resource definition;
launching a configuration portal;
dynamically rendering a user interface in the configuration portal, the user interface being rendered based on the custom resource definition;
receiving configuration inputs on the user interface; and
persisting the configuration inputs into a catalogue database that causes the application to be executed on the medical device based on the configuration inputs.

11. The method of claim 10, wherein the user interface engine provides an interface specification between the application and the configuration portal for obtaining configuration data for the application.

12. The method of claim 10, wherein the application and the configuration portal operate asynchronously with respect to one another.

13. The method of claim 10, wherein the user interface rendered in the configuration portal enables a plurality of configurations for execution of the application.

14. The method of claim 10, wherein the configuration inputs define at least one inbound connection and at least one outbound connection for the application.

15. The method of claim 10, wherein the configuration inputs determine how physiological data captured by a sensor of the medical device is persisted in a database.

16. The method of claim 10, further comprising:
receiving a change to the custom resource definition for the application; and
updating the user interface displayed in the configuration portal based on the change to the custom resource definition, wherein the user interface is updated without modifying the configuration portal.

17. Computer-readable non-transitory storage media embodying software that is operable when executed to:
provide a user interface engine;
receive a custom resource definition of the user interface engine for an application configured for execution on a medical device;
process the custom resource definition;
launch a configuration portal;
dynamically render a user interface in the configuration portal, the user interface being rendered based on the custom resource definition;
receive configuration inputs on the user interface; and
persist the configuration inputs into a catalogue database that causes the application to be executed on the medical device based on the configuration inputs.

18. The computer-readable non-transitory storage media of claim 17, wherein the user interface engine provides an interface specification between the application and the configuration portal for obtaining configuration data for the application.

19. The computer-readable non-transitory storage media of claim 17, wherein the application and the configuration portal operate asynchronously with respect to one another.

20. The computer-readable non-transitory storage media of claim 17, wherein the configuration inputs define at least one inbound connection and at least one outbound connection for the application.
